(19) United States
(12) Patent Application Publication
(10) Pub. No.: US 2012/0052578 A1
(43) Pub. Date: Mar. 1, 2012

(54) GASSING SYSTEM

(75) Inventors: Joerg Kauling, Bergisch Gladbach (DE); Björn Frahm, Lemgo (DE); Andre Pütz, Dusseldorf (DE); Markus Schindler, Hunibach (CH)

(73) Assignee: BAYER TECHNOLOGY SERVICES GMBH, Leverkusen (DE)

(21) Appl. No.: 13/145,823

(22) PCT Filed: Jan. 13, 2010
(86) PCT No.: PCT/EP2010/000124

§ 371 Date: Nov. 16, 2011

(30) Foreign Application Priority Data
Jan. 23, 2009 (DE) 1020090059628

(51) Int. Cl. C12N 5/07; C12N 1/00; C12N 5/04; C12M 1/00; C12M 1/04

(52) U.S. Cl. 435/383; 435/283.1; 435/296.1; 435/420; 435/243

(57) ABSTRACT

A gas-introduction system useful, for example, in biotechnology for supplying cells or microorganisms with oxygen. The gas-introduction system has a bubble column and a distributor. In the vessel there is situated a liquid medium which is to be supplied with gas. A vector is used as transport medium for the gas. The gas is introduced into the bubble column and here taken up by the vector. The vector is applied to the liquid surface via the distributor in droplet form, falls downward in the medium and releases to the medium some of the gas that has been taken up. At the bottom of the vessel is situated a collecting device in which the vector droplets coalesce and pass back into the bubble column. Disclosed are a bioreactor comprising the gas-introduction system and a method for introducing gas into a liquid medium, preferably an aqueous suspension containing cells or microorganisms.

This application is a 371 of International Patent Application No. PCT/EP2010/000124, filed Jan. 13, 2010, which, in turn, claims priority of German Patent Application No. 10 2009 005 962.8, filed Jan. 23, 2009, the contents of both of which are incorporated herein by reference.

The invention relates to a novel gas-introduction system which is usable, in particular, but not exclusively, in biotechnology for supplying animal or plant cells and/or microorganisms with oxygen. The invention further relates to a bioreactor comprising the novel gas-introduction system and also to a method for introducing gas into a liquid medium, preferably an aqueous suspension containing cells and/or microorganisms.

Animal and plant cell culture has established itself in the pharmaceutical industry for producing highly glycosylated therapeutic proteins, monoclonal antibodies and vaccines. Animal cells which, in contrast to microorganisms, do not have a cell wall, are generally distinguished by high shear sensitivity. The oxygen input into the stainless steel reactors used in the pharmaceutical industry is customarily ensured by introduction of coarse gas bubbles. Stirring elements, owing to the restricted shear tolerance of the cells, do not have the function of bubble dispersion, but serve for distributing the gas bubbles, and also mixing the reactor and suspending the cells. The oxygen input and therefore the cell density which can be cultured under these conditions is considerably restricted thereby.

For the gentle oxygen supply of cell cultures, inter alia, membrane gas-introduction is used. As membranes, gas-permeable silicone flexible tubes are wound onto a cylindrical membrane stator and receive flow from a radially-transporting anchor agitator (see, e.g., WO2005/111192 A1). An increase in the exchange surface area and thereby a marked increase in mass transport can be achieved by paralleling the membrane stators.

Other membrane gas-introduction systems (see, e.g. WO85/02195, DE102004029709B4), during the gas introduction, are seated on agitators or baskets which are covered with flexible membrane tubes and are moved in a pendulum manner in the fermentation solution, or are seated on membrane stacks (see, e.g. U.S. Pat. No. 6,708,957 B2) which are pivoted in the fermentation solution. These membrane gas-introduction systems, however, are distinguished in that they can only be converted with restrictions to an industrially relevant scale.

In order to conform to the demand for a rapid and flexible new charging of the production plant, maintaining maximum cleanliness and sterility, designs for disposable reactors are the subject of continuously growing interest on the market. There are a multiplicity of patent applications and patents for using the single-use technology in the field of fermentation technology. In this case, in most systems the mixing and oxygen supply are achieved via bubble gas introduction without further mixing systems being provided (see, e.g., U.S. Pat. No. 5,565,015, WO 98/13469A1, U.S. Pat. No. 6,432,698 B1, WO2005/049785A1, EP1602715A2, WO2005/080544A2). If a relatively high oxygen demand is required in the culture, which cannot be achieved solely by bubble gas introduction, the bubble gas introduction can be combined with a dispersing agitator system (see, e.g., WO2005/104706A2, WO2005/108546A2, WO2005/118771A2) or are overlaid by pump circulation (see, e.g., WO2005/067498A2). The maximum process volume of a unit with bubble gas introduction is currently up to 1000 litres. In systems having conventional agitators but which can also be constructed as disposable systems (see, e.g., WO2005/104706 A2, WO2005/108546A2), process volumes of up to 10 000 l are achieved.

In the case of bubble gas introduction, foam problems can make necessary the use and subsequent complex removal of antifoams in the purification (downstream processing). The stress to cells during the bubble rise, during the surface bursting of the gas bubbles, and in particular in the foam destruction, is problematic in cell culture systems, since the cells can be permanently damaged by the high shear forces introduced in this process. This applies the more so when the bubble gas introduction is combined with a dispersing agitator system, i.e. an agitator system comminuting the gas bubbles. Proteins are released from the destroyed cells, the removal of which proteins during workup can lead to considerable product losses. To maintain acceptable cell viabilities, the oxygen input into the presented bioreactors and therefore also the achievable cell density are restricted. The restricted cell density ultimately reduces the space-time yield of the fermenters and the capacity of the overall plant. Since the precondition for reliable enlargement of scale in most cases must be considered technically as not met, in many bubble gas-introduced disposable reactors the volume enlargement must be achieved by complex paralleling of the systems. If the fermenters are operated as proposed using standard agitator systems, although the volume that is processable increases into the range of the permanently installed plants, the risk of contamination, however, can then only be controlled with comparable technical expenditure, e.g. by using tamped sliding ring seals. However, the large expenditure on equipment and personnel of such installations in a large part again increases the advantages of the disposable concept.

Other disposable systems provide the necessary gas introduction rate to the culture by means of membrane or surface gas introduction. In this case the necessary exchange surface area for the gas transport is provided either via a membrane permeable to the gases that are to be transferred or by a free boundary surface to a gas chamber. Since no direct gas introduction to the cell culture media proceeds, the particle stress must be categorized as relatively low in these reactors.

In U.S. Pat. No. 5,057,429, a system is described in which an inner semipermeable flat pouch that is filled with cell suspension is enclosed by a further pouch which is filled with nutrient solution and enriched with oxygen. Nutrient and oxygen transport are intensified by a tilting motion of the pouches. The maximum process volume of a unit is only a few litres. The oxygen input is considerably restricted by the low oxygen solubility in the charged medium and the comparatively small surface area of the membrane. Compared with standard membrane gas introduction systems (see, e.g., WO2005/111192A1) having specific exchange surface areas in the order of magnitude of 30 m²/m³ in 100 l reactors, in this arrangement only a maximum of 10% of this exchange surface area can be achieved. In both cases, the available exchange surface area decreases, furthermore, in proportion to the enlargement of scale.

Other surface gas-introduction systems likewise operate with a flat pouch which is clamped on a shaking apparatus. The pouch is only partially filled, and so a free surface with a gas space lying thereabove is formed. By means of a tilting motion or eccentric rotary motion, the culture medium is mixed, the nutrients fed are distributed, the cell sedimentation is prevented and the surface is moved (see, e.g., U.S. Pat. No. 6,190,913B1, WO00/66706A1, U.S. Pat. No. 6,544,788B2). In this technology, the culture is supplied with oxygen via the free surface. The motion is always adapted in such a manner that the flow is gentle and the cells are not exposed to excessive shearing. The maximum process volume of a unit is currently 580 litres. Although this technology provides a gentle gas-introduction mechanism, it is restricted in the scale-up to the industrial level. The height of the pouch must be kept approximately constant, in such a manner that a volume increase at constant surface area to volume ratio can only proceed in the two horizontal spatial directions. Scale enlargement can therefore only be achieved via a technically complex paralleling.

In the publication DE102006018824A1, highly promising disposable reactors suitable for surface and membrane gas introduction are described which oscillate in a rotary manner about a central axis and are driven from the outside. The mass transport proceeds via the action of force of rectangular outer walls or of the membranes suspended in the reactor on the inert fluid. The mass transport is intensified via a motion of the liquid surface and/or the relative motion between fluid and membrane. Whereas the rectangular reactor suitable for surface gas introduction, because of the specific exchange surface area that decreases with scale-up, may only be operated in a low-shear manner up to a medium volume, the membrane gas-introduced reactor permits an enlargement of scale at constant shear stress up to industrially relevant production scales of several cubic metres. This has the advantage that, in product development, the process-relevant factors can be kept substantially constant, and that in the transfer of technology from clinic sample manufacturing to production, no bridging studies are required. However, in project planning of the considerable design work and expenditure required for constructing the membrane reactor, and also in comparison with the surface-gas-introduced variant, considerably more complicated installation work for the operating personnel must be accepted.

In the literature, the use of oxygen vectors is recommended as a gentle method for mass transport into cell cultures. Particularly suitable oxygen vectors, because of their chemical stability and an oxygen solubility approximately 20 times greater than water, are the olefinic perfluorocarbons (PFCs). Immiscible with water, these chemicals sediment to the bottom in the cell culture solution owing to their density that is higher than water. Menge et al. (Appl. Microb. Biotech. (2001) 55, 411ff.) describe the use of a gas-liquid-liquid dispersion to be generated by stirring in fungal fermentations in a gas-bubble-introduced stirrer fermenter. Comparatively high feed concentrations of PFCs of greater than 10% are proposed. The high mass fraction, however, makes use of the expensive oxygen vectors uneconomic for a single-use technology. Therefore, the authors propose the reuse of the chemicals, which, however, contradicts the good manufacturing practice (GMP) production principles of restricting the risk of cross contamination. Dispersion of the organic phase by the agitator element is, in addition, impossible because of reasons of an excessive shear stressing of the cell culture. The majority of literature references propose a reactor system consisting of two separate reactors, the fermenter and a gas-introduction reactor, for the oxygen vector, which reactor system is connected to form a loop reactor system by means of a pumped circulation. In Takeshi et al. (Biochem. Engng. J., 8 (2001) 165 ff.), the oxygen-enriched PFC is introduced gently into the cell culture as a falling film. In addition to the mass transport, circulation of the reactor contents via an internal loop is also achieved. The scale enlargement of the design to the industrial production scale, however, is impossible because of the decrease in specific exchange surface area. For proportional scale enlargement, distribution of the oxygen vector in the reactor volume is necessary, as is solved, e.g., in Reschke (Chem. Ing. Tech. 66 (1994) 3, 369ff.) by means of a distributor plate, by which the oxygen vector can be distributed over the fermenter cross section and irrigates through the fermenter as a droplet dispersion. In long-term use, problems could occur owing to blockage of the distributor plates. Pumps and also the additionally necessary external saturation station further increase the complexity and at the same time reduce the robustness of the unit. Since such a unit is unsuitable for the single-use technology, a high expenditure in terms of purification and validation is necessary.

When the above-described gas-introduction systems and bioreactors are used, therefore, despite occasionally highly promising innovative approaches, sacrifices must be accepted in performance, ease of scale-up, long-term stability, robustness and/or operability. An economic benefit, apart from the lack of performance, cannot be ensured in many cases without sufficient scalability.

Therefore, proceeding from the prior art, the object is to provide a gas-introduction system for bioreactors which can be scaled up to the industrial size of 1 m³-10 m³. The gas-introduction system must be usable, in particular in biotechnological, pharmaceutical applications, and have, even on large reactor scales, very good properties with respect to mixing, suspension, solubilization, mass and heat transport, and also combinations thereof. Preferably, it should be simple to handle, and comply with the high cleaning and sterility requirements of the pharmaceutical industry. The use of the gas-introduction system for culturing cells and microorganisms must limit the amounts of waste occurring during production and contribute to increasing process robustness and to increasing the space-time yield.

Surprisingly it has been found that this object can be achieved by a gas-introduction system in which an oxygen vector is transported cyclically between the culture medium and a bubble column within a vessel containing culture medium. In the bubble column the oxygen vector is enriched with oxygen. The oxygen vector is applied in the form of droplets to the liquid surface of the culture medium via a distributor. The droplets fall to the bottom and release at least some of the oxygen to the culture medium. They coalesce in a collecting device at the vessel bottom and from there are fed back to the bubble column. The mixing of oxygen vector droplets and culture medium is preferably intensified by a relative movement of the culture medium with respect to the vessel.

The present invention therefore relates to a gas-introduction system at least comprising
a. a bubble column having at least one intake opening at the lower end of the bubble column,
b. a distributor at the upper end of the bubble column having at least one outlet opening,

characterized in that distributor and bubble column are constructed as hollow bodies and are connected to one another in such a manner that a vector can be introduced through the intake opening into the gas-introduction system and can leave the gas-introduction system again in droplet form via the distributor.

The present invention further relates to a method for supplying a liquid medium in a vessel with gas, characterized in that a vector, in a cyclic process in a bubble column, is enriched by an upwardly directed stream of a gas with at least one component of the gas, is applied via a distributor in the form of droplets onto the surface of the medium, sinks to the bottom in the medium, is collected in a collecting device and is again taken by suction into the bubble column.

### BRIEF DESCRIPTION OF THE DRAWINGS

The invention will now be described in greater detail with reference to the drawings.

FIGS. 1a and 1b are each perspective views of bioreactor embodiments according to the present invention.

FIGS. 2a-2c are each cutaway views of bioreactor embodiments according to the present invention.

FIG. 3 is a perspective view of a bioreactor embodiment according to the present invention.

FIGS. 4a, 4b, 4e and 4f are perspective views and FIGS. 4c, 4d and 4g are cutaway views of bioreactor embodiments according to the present invention.

The gas-introduction system according to the invention and the method according to the invention are preferably used for the introduction of oxygen gas into culture media in bioreactors.

The present invention further relates to a bioreactor at least comprising a vessel for a culture medium, a collecting device for an oxygen vector and a gas-introduction system according to the invention.

A medium is generally taken to mean a substance liquid under the process conditions considered.

A culture medium is taken to mean a suspension of cells (e.g. plant, animal or human) or microorganisms (e.g. bacteria, fungi or viruses) in a liquid medium, preferably in an aqueous medium. It is also conceivable that the cells or microorganisms are present in immobilized form in the culture medium.

A vector is taken to mean a substance liquid under the process conditions considered which is immiscible with the medium or miscible therewith only to a limited extent, has a density higher than the medium under the process conditions considered, and has a higher solubility for a gas than the medium under the process conditions considered.

Perfluorocarbons (PFCs) are particularly suitable as oxygen vectors because of the chemical stability thereof and an approximately 20-times greater oxygen solubility compared with water. Immiscible with water, these substances sediment to the bottom in a cell culture solution owing to their increased density compared with water. Suitable, and preferably used, perfluorocarbons are, e.g., perfluorodecalin, Hostinert or FC40. Their density is virtually twice as high as that of an aqueous culture medium. In comparison with other organic phases such as, e.g., silicone oils, they have the decisive advantage that cells do not accumulate in the organic phase where they would no longer need to be supplied with nutrient media and would be exposed to far too high shear stresses in the bubble column of the gas-introduction system according to the invention.

The gas-introduction system according to the invention acts to supply a medium with a gas. The transport medium used for the gas is a vector. The gas-introduction system according to the invention comprises a bubble column having at least one intake opening which projects into a collecting device. The collecting device is preferably mounted on the bottom of the vessel for the medium. The bubble column is constructed as a hollow, preferably tubular, body. In the bubble column, a vector is enriched with a gas.

Into the bubble column there can be introduced a gas inlet through which gas can be passed into the bubble column. The gas inlet is preferably mounted somewhat above the at least one intake opening of the bubble column. The gas inlet can be introduced into the bubble column from the side. However, the gas inlet can alternatively be introduced into the bubble column from the top, or preferably through an intake opening. Preferably, for improving the momentum exchange, a nozzle is used, through which the gas can be forced into the bubble column in the form of bubbles. As a momentum exchange device, all devices which are reasonably designed in terms of fluid dynamics and which ensure an efficient air-lift pump drive are suitable. Commercially available gas injectors can also be used. The cross section of the bubble column, in the region of the gas introduction, can also have fluid-dynamically advantageous cross-sectional constrictions such as, e.g. venturi profiles. Preferably, the gas inlet in the bubble column is centred in the middle with respect to the cross section and is arranged as an outlet opening or as an annular gap and the outlet cross sections for the gas fed into the bubble column are preferably directed in the direction of the distributor. In the case of relatively large reactor scales, it can also be advantageous to add the gas distributed over a plurality of openings simultaneously over the bubble column cross section. Also, for more efficient usage of the propellant gas, it can be advantageous to ensure the scale enlargement by a numbering-up, i.e. increasing the number of bubble columns.

The gas inlet can be connected to the bubble column; however, it can also be constructed as a separate element which is arranged in a bioreactor according to the invention in such a manner that it projects into the bubble column, e.g. via an intake opening.

It is conceivable to use a frit or the like at the gas inlet in order to match the size of the gas bubbles which are to be introduced into the bubble column to the requirements.

At the upper end of the bubble column, a distributor is mounted. The distributor serves for separating gas and liquid phases, generating droplets and/or distributing the droplets onto the liquid surface of a medium into which the gas is to be introduced.

In a preferred embodiment, the distributor comprises 1 to 20 distributor arms. Particularly preferably, the distributor comprises 2 to 10 distributor arms. The distributor arms are hollow, preferably tubular, bodies which are connected to the bubble column in such a manner that a vector can flow/be forced through the bubble column into the distributor arms. Preferably, the distributor arms are radially arranged about the bubble column. Neighbouring distributor arms, in the case of a central arrangement of the bubble column within the bioreactor, are preferably at an angle of about (360°/n), when n is the number of distributor arms present, i.e. the distributor arms are preferably arranged uniformly distributed around the bubble column. For uniform distribution of the distributor arms in the case of an eccentric arrangement in a corner of the rectangular reactor, the angle should be reduced to 90°/n. The individual distributor arms are at an angle to the longitudinal axis of the bubble column between 110° and 70°, preferably between 100° and 80° (angle of incidence). The diameter of the distributor arms is preferably less than the diameter of the bubble column. The sum of the flow cross-sectional areas of all distributor arms corresponds preferably to about the flow cross-sectional area of the bubble column or is greater than the flow cross-sectional area of the bubble column, in order to decrease pressure drops. Each distributor arm has at least one outlet opening through which a vector can leave the distributor in droplet form. The outlet opening can be mounted on the outer end of a distributor arm. In the simplest case, a tubular distributor arm is constructed to be open at the end. Likewise, it is conceivable to mount one or more outlet openings at the end or along the distributor arm. One or more outlet openings are preferably mounted on the side or the lower side of a distributor arm.

The outlet openings have a diameter in the range from 1 to 100 mm, preferably in the range from 3 to 15 mm. When pumps are used, in particular in combination with a gas preseparation for supporting the transport of the oxygen vector, in addition, the use of nozzle systems having smaller outlet cross-sectional areas and also the use of a centrally arranged nozzle for generating conical liquid films is conceivable.

In addition to the above-described star-shaped or ray-shaped arrangement of distributor arms, it is also conceivable to construct the distributor in a ring shape or spiral shape. In such an embodiment, outlet openings are preferably arranged uniformly distributed over the ring or the spiral. Further shapes of the distributor are conceivable. The distributor is preferably matched to the shape of the vessel for the medium. The distributor is likewise preferably matched in position in relation to the vessel. The distributor is preferably constructed in such a manner that it distributes the vector in the form of droplets as evenly as possible onto the surface of the medium.

Distributor and bubble column can be made from one piece; however, they can also be fabricated from different pieces and connected to one another via a reversible or irreversible connection. Preferably, bubble column and distributor are fabricated from different pieces. Preferably, bubble column and distributor are connected to one another via a reversible connection. In a preferred embodiment, the distributor and the bubble column are placed one within the other for connection.

Distributor and bubble column can be fabricated, e.g., of metal, plastic or glass. Distributor and bubble column are preferably constructed as disposable articles made of plastic in order to ensure a very high level of process safety with respect to cleaning and sterile technology. Suitable plastics are, e.g., PVC, polyolefins, polyesters, polyethylene, polypropylene, PEEK, inter alia, and also combinations thereof.

The method according to the invention for gas introduction into a medium in a vessel containing a gas is characterized in that a vector is transported between medium and a bubble column in a cyclic process. In the bubble column the vector is enriched with at least one component of the gas. For that purpose the gas is forced into the bubble column in the form of bubbles. The dispersion of vector and gas bubbles ascends in the bubble column owing to a reduced density above and passes into a distributor. In the distributor the vector and the gas for the most part separate. The gas leaves the distributor and passes into the head space of the vessel where it can be removed by suction. The vector enriched with gas (or a component of the gas) is applied to the liquid surface of the medium in droplet form via the distributor. The vector droplets fall downward in the medium and release the at least one component of the gas to the medium at least in part. The drops coalesce in a collecting device from where they pass back into the bubble column. The mass transfer between the vector droplets and the medium is supported by a relative movement of the medium with respect to the vessel.

The gas-introduction system according to the invention and the method according to the invention permit a very simple, easily scalable and extremely gentle supply of a medium with gas and are therefore particularly suitable for supplying biological cultures—preferably human, animal or plant cells—with oxygen. The present invention therefore also relates to the use of the gas-introduction system according to the invention and the method according to the invention in a bioreactor for introduction of oxygen gas into the culture medium. A bioreactor is taken to mean a system which serves for propagation and/or growth and/or storage of living cells and/or microorganisms.

In addition to supplying cells or microorganisms with oxygen, the gas-introduction system according to the invention and the method according to the invention also serve for removing gaseous metabolic products such as, e.g. carbon dioxide. As transport medium for oxygen and/or gaseous metabolic products, an oxygen vector is used.

The method according to the invention for supplying cells or microorganisms in a bioreactor according to the invention with oxygen is characterized in that an oxygen vector is enriched with oxygen in a cyclic process in a bubble column into which an oxygen-containing gas is introduced, ascends upwards in the bubble column, is applied to the liquid surface of the culture medium via a distributor in the form of droplets at the top end of the bubble column, falls to the bottom of the vessel in the liquid, collects in a collecting device and from there is again taken by suction into the bubble column.

A reservoir of the oxygen vector is present in a collecting device in the bottom region of the bioreactor. Via at least one intake opening which extends into the collecting device and is sufficiently covered over by the single-phase oxygen vector, the organic phase is introduced into a bubble column. For this purpose, just above the intake opening, an oxygen-enriched gas is forced into the bubble column preferably via an upwardly directed nozzle piece. For the transport, depending on the type and density of the oxygen vector or the intended recirculation volumetric flow rate, comparatively high superficial gas velocities of 0.01-10 m/s, preferably 0.1-3 m/s, are required in the bubble column, which may be achieved with moderate gas volumetric flow rates using small bubble column diameter to vessel diameter ratios of 0.01<d/D<0.1. In the case of numbering-up, the inner diameter of the bubble columns d should be in the range 3 mm<d<50 mm, and preferably between 5 and 10 mm. At the optimum operating point, for the transport of the oxygen vector exactly as much gas is introduced into the gas inlet of the bubble column as is required for oxygen saturation or carbon dioxide stripping of the organic phase. Since this optimum operating point is not always achievable and generally a gas excess is required for liquid transport, it can be expedient for cost reasons to recirculate some of the exhaust gas to the propellant gas stream. In order to restrict the complexity of the method and to meet its demands for robustness and sterility, it is advisable to carry out the gas circulation likewise using static non-invasive elements. Self-priming gas injectors which are driven by the gas feed stream are highly suitable for this task. It can also be expedient to support the transport of the oxygen vector additionally by a pump connected between bubble column and distributor, preferably an externally arranged non-invasive peristaltic pump or an internally driven centrifugal pump having suitable sterile couplings, e.g. magnetic or steam-covered sterile couplings. For this purpose, under some circumstances, separate gas preseparation upstream of the pump would be advisable.

Furthermore, because of the spatial separation of the culture medium, the high gas-introduction intensities in the bubble column do not show a disadvantageous action on the shear-sensitive biological culture. At the top of the bubble column, the gas-oxygen vector dispersion is passed through the distributor arms to the outer walls of the vessel. The cross-sectional areas of all internals including the distributor arms can be chosen to be of a size such that blockage of the lines can be excluded. At the exit of the distributor arms, the oxygen vector is applied to the liquid surface of the culture medium as a droplet dispersion having a comparatively low droplet size in the lower mm range. Droplet sizes that are too small are avoided by the dimensioning of the outlet cross-sectional areas. These droplets run the risk of being discharged from the vessel together with the gas stream and would therefore have to be replaced. Astonishingly, the experiments have found that the droplets, on passage through the liquid surface of the culture medium, can encapsulate a gas bubble.

In this connection, the use of surfactants such as, e.g. Pluronic, is advantageous, which, in addition, prevent attachment of the organic cells to the organic phase, is advantageous. This conjugate of gas and liquid phase has the great advantages of an additionally enlarged gas exchange capacity and a decreased falling rate of the liquid droplets. In addition, this results in an increased exchange surface area for mass transport between organic phase and the culture medium.

Preferably, a movement unit is used which generates a relative movement of the culture medium with respect to the vessel. By means of the movement, a current is transferred to the culture medium which, not only at the liquid surface, but also within the vessel, prevents premature coalescence of the oxygen rector droplets and in addition reduces the liquid-side mass transport resistance on the droplet exterior. In addition, the organic phase, despite the point-source feed sites of the distributor, is gently distributed over the reactor cross section and thereby meets an essential condition for effective transfer of scale.

The droplet dispersion of the oxygen vector sediments in the culture medium. The droplets having arrived at the container bottom are collected in a collecting device which is preferably configured as having one or more bottom recesses which are conical, pyramidal or directed towards one or more reactor corners, and coalesced to form a continuous phase.

The amount of oxygen vector to be stocked in the gas-introduction system according to the invention, at least comprising a bubble column and a distributor, is very small owing to the small dimensions of the elements. Therefore, even a small feed rate of oxygen vector set in the low % by volume range of 0.3% by volume to 10% by volume, preferably 0.5 to 2% by volume, based on the volume of the culture medium is sufficient in many cases for an adequate oxygen feed. Despite the comparatively high cost of chemicals, this offers the possibility of using the oxygen vector without the risk of cross contamination for the single use, whereby this technology is suitable, inter alia, for the structure of disposable bioreactors. The relative movement of the culture medium, in addition to the gentle distribution of the droplet dispersion, achieves the reduction of the liquid-liquid mass transport resistance (oxygen vector/culture medium), likewise the gentle mixing of the culture medium and also suspension of the cells, immobilized cells or microorganisms.

The present invention further relates to a bioreactor. A bioreactor according to the invention comprises a vessel for receiving cells or microorganisms which are usually in an aqueous suspension. The bioreactor according to the invention further comprises the gas-introduction system according to the invention which serves for supplying the cells or microorganisms with oxygen and also transporting away from the culture medium gaseous metabolic products. The bioreactor according to the invention further comprises at least one collecting device into which a bubble column of the gas-introduction system at least in part protrudes.

Since the oxygen vector, owing to its higher density, falls to the bottom in a culture medium, the collecting device is arranged preferably at the bottom of the bioreactor vessel. In a preferred embodiment of the bioreactor according to the invention, at the bottom of the bioreactor a recess is introduced. The recess is preferably constructed so as to be tapering downwards. The recess is constructed so as to be, e.g., conical, tetrahedral, pyramidal, or as inclined planes in one or more reactor corners. Further shapes are conceivable. The recess can be mounted in the middle or at one side of the vessel bottom. The bubble column protrudes at least in part into the recess. The bubble column can be fastened via a bottom mount at the bottom of the bioreactor, on side mounts at one or more sides of the bioreactor or on a top mount at the top of the bioreactor. It is likewise conceivable to couple the bubble column outside the reactor via connecting pieces to the bottom recesses. In one embodiment as a disposable reactor, by means of an external coupling of the bubble column, advantages can result in the packaging. For space-saving packaging, it would be advantageous to construct the bubble column unit itself likewise from flexible materials or a combination of rigid and flexible elements which are unfolded to form a vertical column for startup of the bioreactor. Also, a sterile bubble column made up of rigid elements could be first coupled to the bioreactor via sterile connections immediately before startup. In the case of in-situ steam sterilizable or externally autoclaved systems it is conceivable to construct the bubble column to be mounted so as to be able to rotate within a hollow shaft. The shaft is preferably connected to the external drive via a sterile shaft coupling, preferably a magnetic coupling or sliding ring seal.

One or more gas inlets in the bubble column are supplied with an oxygen-containing gas, e.g., via a feed port at the top of the bioreactor and/or a flexible tube between feed port and gas inlet. It is likewise conceivable to introduce the gas inlet into the bubble column via a port at the bottom of the reactor.

The bubble column is preferably arranged at low-lying points in the middle in the bioreactor and/or at the sides thereof within or outside the reactor corners. The distributor is preferably mounted in the head space of the bioreactor above the liquid surface of the culture medium. The spacing between the outlet openings of the distributor and the liquid surface is preferably in the range between 0.01×D to 0.3×D, or preferably between 10 mm and 500 mm, preferably between 20 mm and 100 mm. The height difference figures refer here to the completely filled reactor. In the case of a static installation of the gas distributor, this spacing can be many times the optimum spacing at the start of fermentation, e.g. after inoculation, at low fill levels, and so effective transport of the oxygen vector is no longer ensured. For coverage of the oxygen demand in the propagation phase, in the case of a cell concentration that is to be limited by suitable feeding strategy, surface gas introduction to the reactor moved in an oscillating manner is of a completely adequate amount. Connecting in the oxygen vector gas introduction is advisable in this case after reaching a minimum cell count which is not sought until after the reactor is filled to the optimum fill level.

It is likewise conceivable to position the distributor so as to be variable in height in the reactor. The position of the distributor can be implemented, for example, mechanically by a level-controlled controller or a floating mount on the liquid surface. Also, a pump installed between bubble column and distributor can ensure operation of the reactor at low fill levels.

The distributor is preferably matched to the geometry of the bioreactor. When radially arranged, tubular distributor arms having open ends are used, they sweep over between 30° and 90° of the half reactor cross section.

The bioreactor according to the invention is constructed, in particular, as a disposable reactor which can be discarded after use is complete. For this purpose, the reactor vessel can be made of a stable, preferably multi-layer plastic or of a plastic applied to stabilizing net structures and supporting the intended basic processing operation. Preferably, the reactor vessel is connected to a housing that matches at least in part the shell shape of the reactor. The reactor is preferably constructed of single layer or multilayer film materials. These are constructed in such a manner that the release of film contents (extractables or leachables) is reduced to a minimum level. In the region of the reactor walls coming into contact occasionally or permanently with the oxygen vector, it can be necessary to produce these walls from special materials or to laminate or coat them with special impermeable layers.

In a preferred embodiment, the bioreactor according to the invention is combined with a movement unit. The movement unit serves for generating a relative movement of the culture medium with respect to the reactor vessel. This relative movement promotes the mixing of the oxygen vector droplets and of the culture medium. It improves the mass transport between the oxygen vector droplets and the culture medium.

In a preferred embodiment, the movement unit is a drive unit to which the reactor vessel is coupled. The bioreactor can also put into an oscillating rotary motion around a stationary, preferably vertical, axis of the reactor, by the drive unit. The oscillating rotary motion comprises a reversal of motion. Owing to inertia, the culture medium lags behind the movement of the bioreactor, which leads to a relative movement of the culture medium with respect to the vessel, which causes good mixing of the culture medium and good distribution of the oxygen vector droplets within the culture medium.

By means of a suitable shell shape of the bioreactor and/or internals within the vessel, the power input into the culture medium can be increased and thereby the mixing improved. Preferably, the bioreactor, in a preferred embodiment, therefore has at least in part an angular cross section, preferably two-cornered to octagonal, particularly preferably triangular to quadrangular cross section, perpendicular to the axis of rotation. In this case, the cross-sectional shape can also change along the height of the reactor in an axial direction (along the axis of rotation). Thus, the reactor can be constructed, for example in the upper region, cylindrically or square, and in a lower region rectangular, square, pyramidal, tetrahedral, etc. By means of a rotary motion of the reactor thus constructed, liquid currents can be generated in the culture medium.

Preferably, the bioreactor is positively coupled to the drive unit in such a manner that the acceleration and braking of the rotary movement proceeds with a substantially constant angular acceleration or deceleration. As a result, the rotational speed of the reactor changes linearly with time in each movement phase of the rotary oscillation. Intermediately connected control modules are not required in the case of this simple reactor movement, and so, for example, according to a preferred embodiment, for implementing the oscillating movement, a pendulum-type gearing can be used. By this means, it is possible to reduce drastically, e.g., the release of electromagnetic rays, which can cause, e.g., interference with sensors. In particular, owing to the constant angular acceleration in each phase of a rotary oscillating movement, instantaneous peak values of the hydrodynamic shear forces on suspended particles (e.g. animal cells) are kept comparatively lower than in other movement forms of the reactor.

It is also conceivable, instead of a rotary oscillating movement, to carry out a pendulum-type or tilting, or a combined rotary/pendulum-type and/or tilting movement. The critical factor is that the movement proceeds discontinuously, i.e. the vessel carries out accelerated or braked movements in which the culture medium, owing to inertia, lags behind the movement of the vessel. It is known to those skilled in the art how a corresponding movement unit needs to be designed and coupled to the vessel in order to carry out a corresponding movement.

In a further preferred embodiment, the bioreactor according to the invention has a gas-introduction unit at the bottom of the vessel, which gas-introduction unit acts as movement unit. This gas-introduction unit comprises at least one gas-introduction tube which is preferably mounted in the bottom region of the vessel. The gas-introduction tube comprises a gas inlet, via which the gas-introduction tube can be supplied with gas. The gas-introduction tube comprises in addition openings through which gas can be forced from the gas-introduction tube into the medium. Depending on requirements, the openings should be constructed in such a manner that fine- or coarse-bubbled gas introduction is possible.

Fine gas bubbles are taken to mean gas bubbles which have a slight tendency to coalescence in the culture medium used. Suitable means for fine-bubbled gas introduction are, for example, special sintered bodies made of metallic or ceramic materials, filter plates or laser-perforated plates, which have pores or holes having a diameter of generally less than 15 μm. In the case of low superficial gas velocities of less than 0.5 mh⁻¹, very fine gas bubbles are generated which have a low tendency to coalescence in the media usually used in the cell culture.

Coarser bubbles are generated by correspondingly larger holes. The gas introduction generates a circulation vortex which moves the culture medium relative to the vessel and generates good mixing.

In the case of the above designs, a shaft passage can be dispensed with, as is the case with pumps or complicated, blockage-susceptible distributor plates. Apart from a possible compressor for gas feed and possibly a drive for the preferably oscillating motion of the vessel, no further installations that need to be externally driven and are connected to the product (e.g. agitators or pumps) are required for transporting the media. In the case of a restricted gas flow rate, cultures can also be carried out in a very low-shear manner with direct gas introduction.

The latter is, in particular, of critical importance in the case of shear-sensitive cultures with animal cells which must be supplied with oxygen, e.g. during fermentation. Because of the high shear forces, here, excessively intense bubble gas introduction is frequently not used, and so generally the relatively low-shear method according to the invention by means of gas-enriched organic oxygen vectors is used.

In a further preferred embodiment, the relative movement of the culture medium with respect to the vessel is generated by means of an agitator device within the vessel as movement unit.

Preferably, in this case agitator device and bubble column are combined with one another: via a sterile coupling at the top of the bioreactor, a shaft is introduced into the bioreactor. On the shaft are mounted the bubble column, the distributor and stirrer blades. The gas inlet within the bubble column is introduced preferably via the bottom of the bioreactor. The shaft is driven via a motor which moves the distributor, bubble column and stiffer blades. The movement can be continuous or discontinuous. It can be oscillating. Additional internals within the bioreactor can be used as baffles which promote mixing.

The bioreactor permits work with culture media in the filled state at a ratio of liquid height to average diameter of 0.2-3.0, preferably 0.6-1.8, and particularly preferably 0.8-1.2. Of course, the bioreactor can also be operated with partial fillings in the growth phase. The head space above the liquid in the filled state is about 10-30% of the liquid height. Owing to the low fill heights compared with commercially conventional bioreactors, tilting moments caused, e.g., by imbalance, can be reduced and, despite an installation space requirement which can be achieved without problem even on a large scale, a possibility of operation from the top is ensured. Compared with the slim reactors introduced in biotechnology, by means of a broad reactor design, this offers the possibility, in the accommodation of the reactors, to avoid expensive tall buildings in favour of installation in cheaper hall-shaped plants.

The bioreactor according to the invention can be constructed as a heat-sterilizable reactor, preferably made of stainless steel or glass, or preferably as a disposable reactor made of plastic.

In a preferred embodiment, the bioreactor according to the invention has at least one sensor intended for single use, with the aid of which in particular a pH and/or an oxygen concentration and/or the temperature of the reactor contents can be detected.

The invention will be described in more detail hereinafter with reference to examples, but without being restricted thereto.

### EXAMPLES

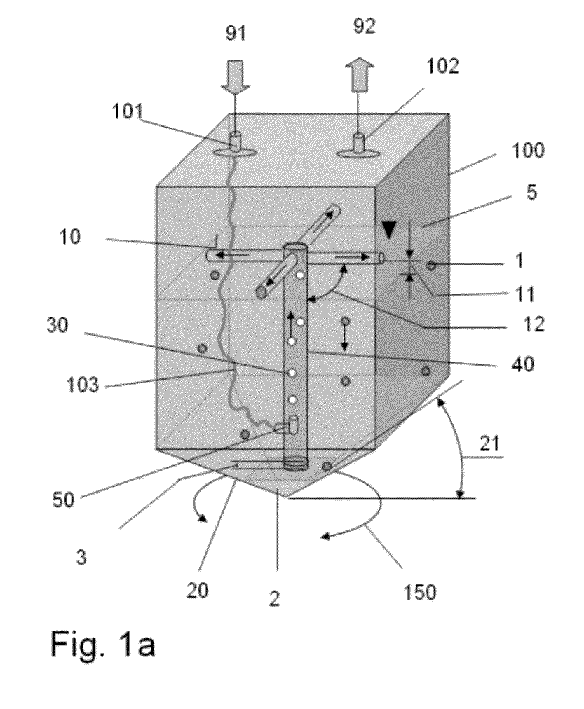

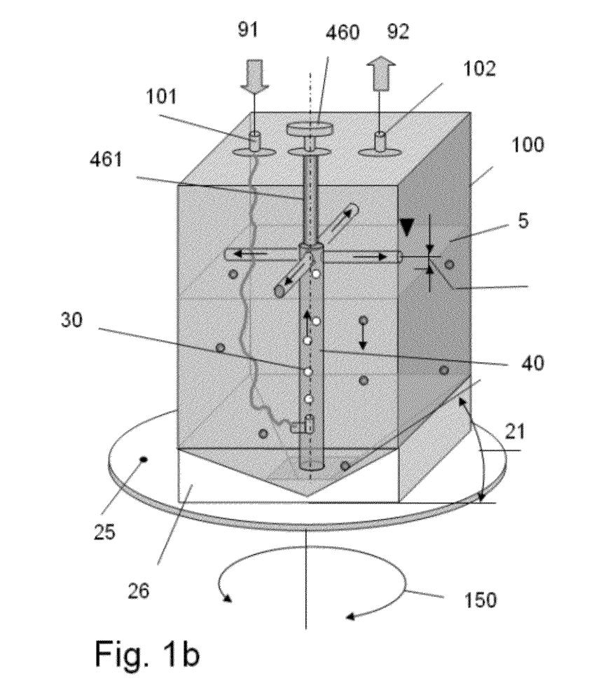

In FIG. 1a, a preferred embodiment of a bioreactor according to the invention is shown. The bioreactor comprises a vessel (100), in the centre of which a gas-introduction unit according to the invention is installed. On the vertical axis of the bioreactor, a bubble column (40) is arranged which is equipped with a gas nozzle (50) for gas distribution. The bubble column (40) which is open at the bottom protrudes into the pyramidal bottom (20) of the rectangular reactor. There it falls below the fill level of the oxygen vector in the reservoir (2) by a covering height (3). The oxygen vector is deposited there as a consequence of its density which is greater than the fermentation medium, wherein the droplets (1) fuse or coalesce to form a continuous phase. The bottom angle of incidence (21) should be dimensioned to be sufficiently large in order to ensure ready accumulation of the droplets (1) of the oxygen vector and also risk-free removal thereof from the reservoir (2) in the case of a sufficient liquid coating (3) of the intake opening of the bubble column (40), without a short-circuit stream for the fermentation medium being formed. Avoiding a short-circuit stream introduced into the bubble column is particularly important in order to prevent destruction of the cells in the bubble column (40) operated with very high shear rates. The covering (3) should be minimized to minimize the requirement of oxygen vector for cost reasons. At low dynamic vacuums in the suction zone, a few centimetres in the region of the coating height (3) are sufficient for the height of the liquid level (5) of the aqueous phase of Δh/H=0.01-0.1 in order to prevent short-circuit currents. Gas is introduced to the organic phase and/or the oxygen vector via the nozzle (50) which is connected via a flexible tube (103) via feed ports (101) to the oxygen-enriched gas (91) in order to enrich this with oxygen and strip out the carbon dioxide taken up with the fermentation solution. The gas introduction is intensive in such a manner that the median density of the gas and oxygen vector can surprisingly be lowered to far below the median density of the fermentation solution in such a manner that the oxygen vector can even be lifted up to a distance (11) above the liquid level of the fermenter chamber. The superficial gas velocities for transporting the oxygen vector are in the range from 0.01 to 10 m/s, preferably 0.1-3 m/s. The oxygen vector is then applied to the liquid surface as droplet suspension via the distributor arms (10). Favourable feed positions, in the case of a reactor which is driven in an oscillating manner from outside, are situated on the perpendicular horizontal axes to the container walls, because at these positions a particularly good movement of the liquid surface proceeds, and a favourable distribution of the droplet suspension in the reactor is thereby made possible.

Surprisingly, the droplets (1), on entry thereof into the fermentation solution in the presence of suitable surfactants (e.g. Pluronic) enclose a gas bubble. The reversible conjugate of gas bubble and oxygen vector possesses the advantage of a reduced density difference from the culture medium with the consequence of an improved homogenizability and an increased gas fraction and an increased exchange surface area. For improved bubble dispersion, it can be expedient to reduce slightly the angle of incidence (12) of the distributor arms (10). Favourable angles of incidence (12) are between 90° and 70°. Favourable feed positions of the distributor tubes are between 0.45 to 0.95-times the reactor width D.

FIG. 1b shows how the bioreactor described in FIG. 1a can be constructed as a disposable reactor in a plastic pouch. In this case, the bubble column (40) can be mounted from the outside via a connection element (461) having a mount (460) which passes through the plastic pouch (100) and is centred on the vertical axis. The bottom shape results from placing the readily deformable plastic container onto a correspondingly shaped bottom element (26) which is fastened to the disc (25) that is moved in a rotating oscillating manner.

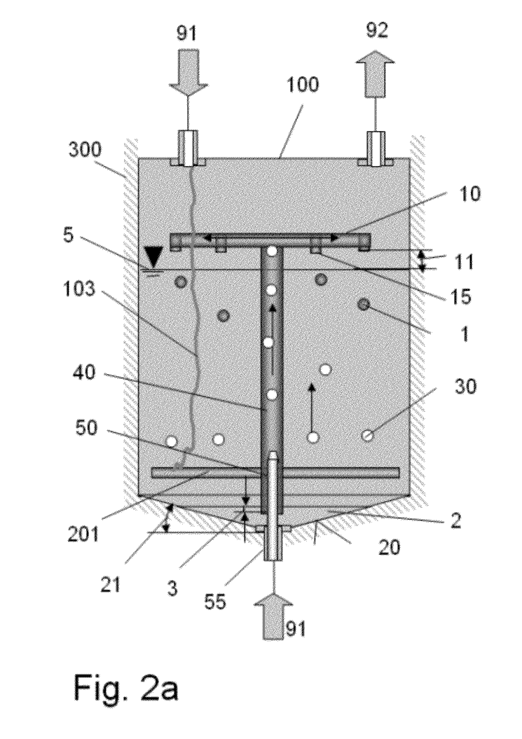

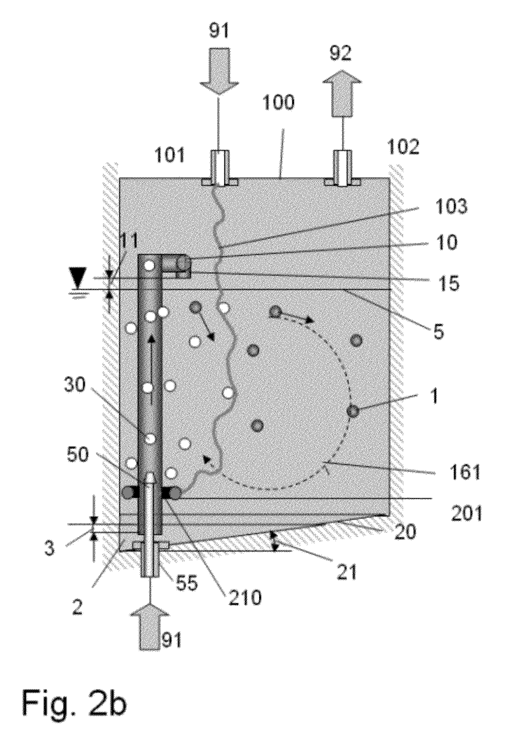

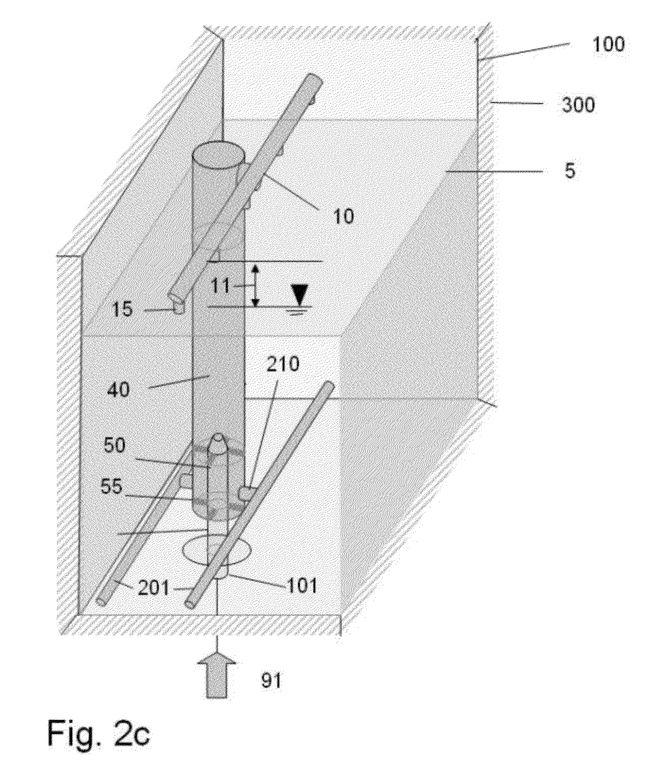

FIG. 2 shows how a gas-introduction system according to the invention can be combined in a bioreactor with a bubble gas-introduction-induced liquid movement. The bubble column (40) can be mounted via a nozzle tube (50) which is bearing-mounted in the bottom mount (55) and is supplied with gas through this. The oxygen-enriched oxygen vector is introduced into the fermenter chamber via a distributor tube (10) via the outlet openings (15) at a distance (11) above the liquid level (5). The oxygen vector which is applied to the liquid surface (5) as a droplet dispersion is mixed by a large-volume liquid vortex (161). The liquid vortex (161) which is shown in side view in FIG. 2b is driven by the linear bubble gas introduction via the gas-introducer (201). This is supplied with oxygen-enriched gas (91), for example by means of a flexible tube (103) from the outside via the port (101). For low-energy operation of the liquid vortex, expediently, an asymmetric positioning of the linear gas-introducer (201) is selected in such a manner that the height and the width of the liquid vortex (161) are as far as possible of the same size. The gas-introduction tubes may be anchored externally relatively simply to the bubble column as shown in FIG. 2c by means of the fastening elements (210). The shape of the bottom (20) may be expediently established by selecting a suitable angle of incidence (21) in such a manner that the accumulation and transport of the oxygen vector to the bubble column (40) is facilitated and, in the case of sufficient covering (3), the oxygen vector can be introduced into the bubble column (40), avoiding a short-circuit stream to the fermentation solution.

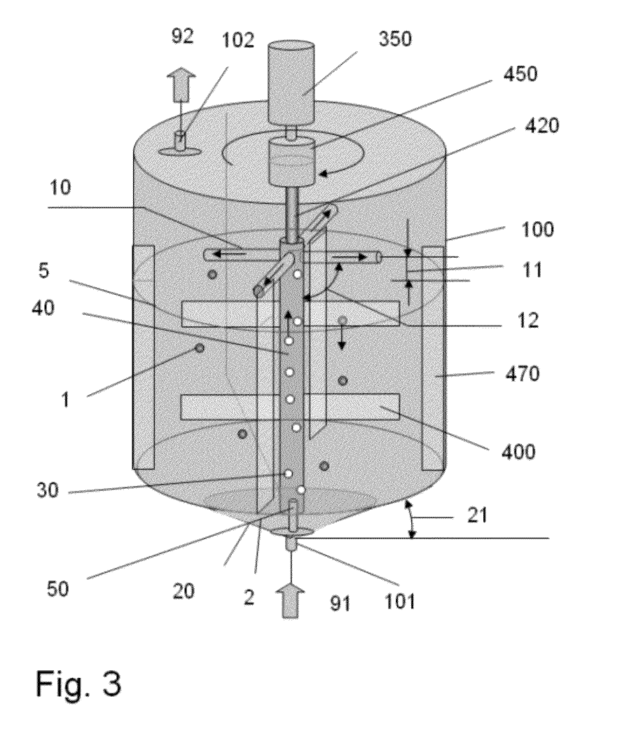

FIG. 3 shows how the gas-introduction system according to the invention can be combined in a bioreactor with an agitator. On the stirrer shaft (420) connected via a sterile coupling (450) to the motor (350), the bubble column (40) for the oxygen vector can be fastened in axially symmetrical manner. The bubble column (40) reaches on the suction side into the reservoir (2) for the oxygen vector and is there sufficiently covered by the oxygen vector phase. The oxygen vector is accumulated and coalesced in the conical bottom (20) having the angle of incidence (21) of the reactor. The gas-introduction nozzle (50) that is preferably connected to the port (101) can be introduced in a contact-free manner into the slowly rotating bubble column (40), for example from the bottom via the conical or pyramidal tip or from the centre of a domed or round bottom. On the outside of the bubble column, at one or more heights, one or more stirrer paddles (400) that are set perpendicular to or facing the vertical axis are fastened, which ensure a gentle distribution in the fermentation medium of the oxygen-enriched droplet dispersion of the oxygen vector that is added above the liquid surface (5). In this case it can be advantageous to place one level of stirrer blades (400) just below the liquid surface and to feed the droplet dispersion onto the liquid surface (5) via the trailing region of the stirrer paddles, in order in this manner to make possible particularly intensive droplet distribution in the fermentation medium. The reactor (100), for preventing rotary currents, can be equipped with baffles (470). Instead of the distributor arms (10), conical or cylindrical distributor internals or distributor plates are also suitable for applying the oxygen vector.

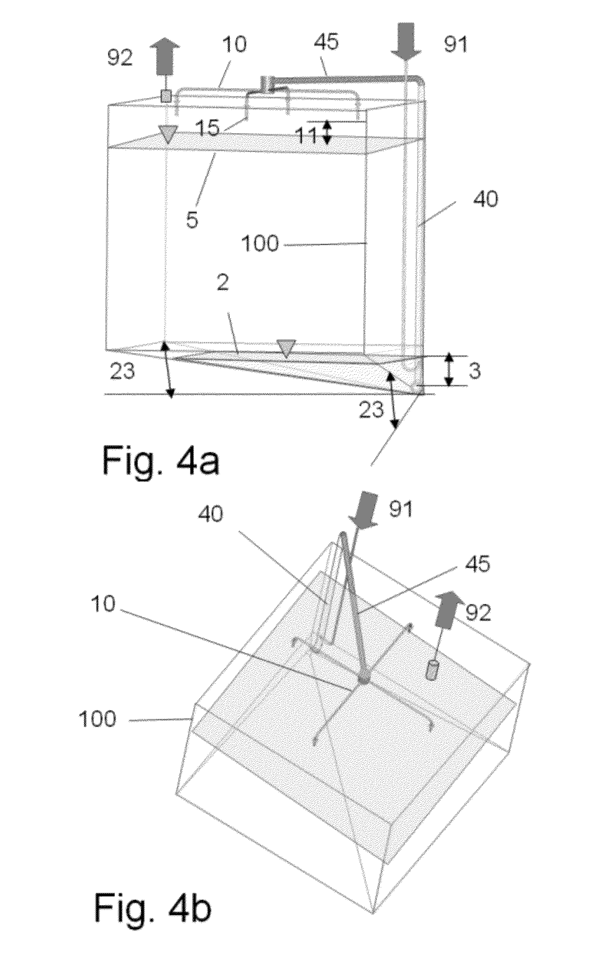

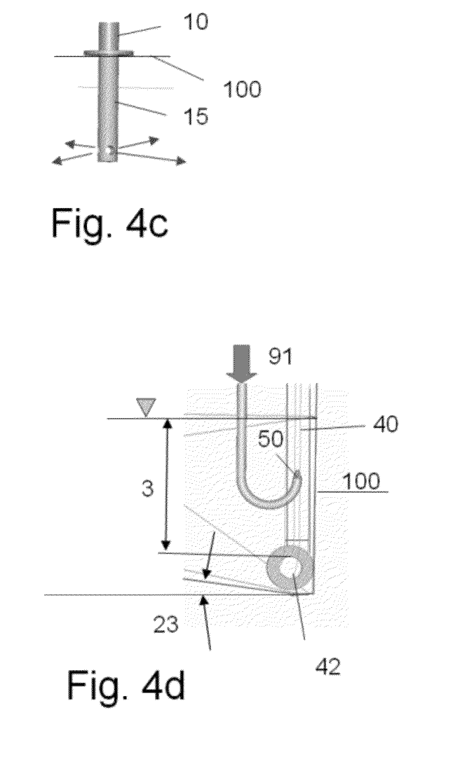

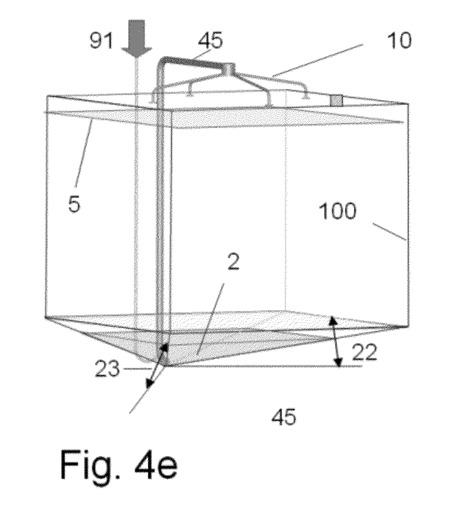

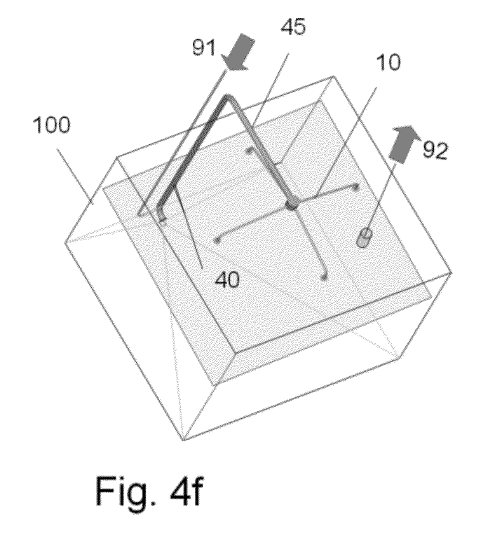

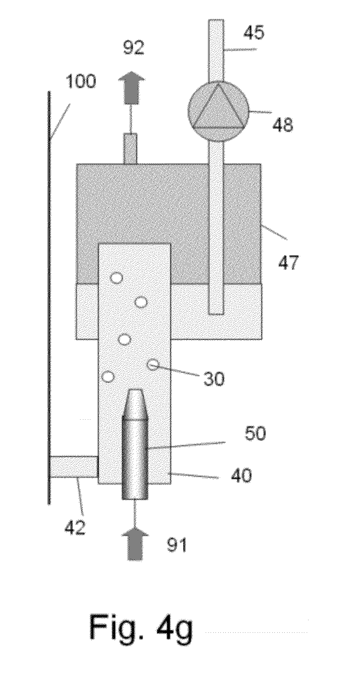

In FIGS. 4a to 4g, a reactor is shown having external gas introduction and external transport of the oxygen vector. This produces the advantage of a reduced construction expense and packing volume requirement, which, in particular, markedly simplifies the use of the reaction vessel (100) as a disposable reactor. As FIG. 4d shows, the oxygen vector, in the region of the lowest container position of the bottom, is transported to the bubble column (40) via an external connection (42) installed in the reactor vessel (100). In the bubble column (40), the gas is fed (91) via the nozzle (50). As shown in the views for front view and plan view of a reactor configuration having externally gas-introduced oxygen vector in FIGS. 4a and 4b and also FIGS. 4e and 4f, the bubble column (40) can be attached directly to the external transfer line (45) which leads to the distributor arms (10). After passing again through the container wall of the reaction vessel (100), the oxygen vector can then be distributed onto the surface (5) by means of the distributor nozzles (15). In the reaction vessel in FIGS. 4a and 4b, the oxygen vector is taken off at a corner of the reaction vessel (100) having constant bottom angles of incidence (23). In FIGS. 4e and 4f, the takeoff proceeds on a container axis having differing bottom angles of incidence (22) and (23). In a preferred use of the external oxygen enrichment of the oxygen vector which is shown in FIG. 4g, gas introduction and transport of the oxygen vector are decoupled. In this manner the required amounts of gas may be reduced to the requirement for the oxygen enrichment, whereas the feed stream through the hermetic pump (48) can be set independently of the gas volumetric flow rate. Hermetically acting pumps (48) which are suitable are, for example, peristaltic pumps which can be connected to the process without hazard to the sterility by placing in the sterilized flexible pump tubes to the fermentation. The flexible pump tubes are part of the external transfer line (45) which is connected to the gas separator (47).

### REFERENCE SIGNS

1 Oxygen vector droplets
2 Oxygen vector reservoir
3 Covering height
5 Liquid surface of the culture medium
10 Distributor
11 Distance of the outlet openings of the distributor to the liquid surface of the culture medium
12 Distributor angle of incidence
15 Outlet opening on the distributor
20 Vessel bottom
21 Bottom angle of incidence
22 Bottom angle of incidence
23 Bottom angle of incidence
25 Disc
26 Bottom element
30 Gas bubble
40 Bubble column
45 Transfer line
47 Gas separator
48 Hermetic pump
50 Gas inlet
55 Bottom mount
91 Gas feed
92 Gas removal
100 Vessel
101 Gas feed port
102 Gas removal port
103 Flexible tube for gas supply
150 Rotationally oscillating movement
161 Circulation current
201 Gas-introduction tube
210 Fastening element
300 Container wall
350 Motor
400 Stirrer blade
420 Stirrer shaft
450 Sterile coupling
460 Mount
461 Connection element
470 Baffle

1. Gas-introduction system for supplying a liquid medium in a vessel with gas, said gas-introduction system at least comprising:
a. a bubble column having at least one intake opening at the lower end of the bubble column,
b. a distributor at the upper end of the bubble column having at least one outlet opening,
wherein distributor and bubble column are constructed as hollow bodies and are connected to one another in such a manner that a vector can be introduced through the intake opening into the gas-introduction system and can leave the gas-introduction system again in droplet form via the distributor.

2. Gas-introduction system according to claim 1, wherein the distributor has 2 to 10 distributor arms which extend radially out from the bubble column and are at an angle of 110° to 70° to the longitudinal axis of the bubble column.

3. Gas-introduction system according to claim 1, wherein the distributor is constructed to be ring- or spiral-shaped and has outlet openings on the distributor bottom that are distributed uniformly over the length of the distributor.

4. Method comprising supplying cells or microorganisms with oxygen using a gas introduction system according to claim 1.

5. Bioreactor at least comprising a vessel for receiving a culture medium, a gas-introduction system according to claim 1, and a collecting device, wherein the intake opening of the bubble column projects into the collecting device situated at the bottom of the vessel.

6. Bioreactor according to claim 5, wherein the collecting device is formed by a downwards tapering bottom of the bioreactor having a conical, tetrahedral or pyramidal shape.

7. Bioreactor according to claim 5, additionally comprising a gas inlet which is introduced into the bubble column centered at the middle above the intake opening of the bubble column or via an annular gap at the periphery.

8. Bioreactor according to claim 5, wherein an enrichment of the vector with gas and a transport of the enriched vector into the distributor proceed decoupled from one another, in that the transport of the vector is performed or supported by a pump.

9. Bioreactor according to claim 5, additionally comprising a movement unit which generates a relative movement of the medium with respect to the vessel.

10. Bioreactor according to claim 9, wherein the movement unit is constructed as a drive unit which is coupled to the housing and generates a discontinuous movement of the housing.

11. Bioreactor according to claim 10, wherein the movement unit is constructed as an agitator, wherein a stirrer shaft is conducted through the top of the bioreactor, on which shaft are fastened directly or indirectly the distributor, the bubble column and stirrer blades.

12. Bioreactor according to claim 11, wherein the movement unit is constructed as a gas distribution unit for bubble gas introduction which generates a circulating flow of the culture medium within the vessel.

13. Method for supplying a medium in a vessel with gas, comprising enriching a vector, in a cyclic process in a bubble column, by an upwardly directed stream of a gas with at least one component of the gas, applying via a distributor in the form of droplets or jets onto the surface of the medium, sinking to the bottom in the medium, collecting in a collecting device and again taking by suction into the bubble column.

14. Method according to claim 13, wherein, by means of a movement unit, a relative movement of the medium with respect to the vessel is generated, which movement effects a better mixing of vector droplets and medium.

15. Method according to either of claims 13, wherein the vector used is a perfluorocarbon, the gas used is an oxygen-containing gas, and the medium used is an aqueous culture medium containing cells or microorganisms.

16. Method according to claim 13, wherein a surfactant is used which prevents the attachment of cells or microorganisms to the vector.
